(19) United States
(12) Patent Application Publication
(10) Pub. No.: US 2007/0224148 A1
(43) Pub. Date: Sep. 27, 2007

(54) Beta-(1,3)-Beta-(1,4)-Glucan as Carrier for Chemical Substances

(75) Inventor: Joachim RÖDING, Hamburg (DE)

Correspondence Address: ROYLANCE, ABRAMS, BERDO & GOODMAN, L.L.P., 1300 19TH STREET, N.W., SUITE 600, WASHINGTON,, DC, 20036, US

(73) Assignee: SYMRISE GMBH & CO. KG, Holzminden (DE)

(21) Appl. No.: 11/573,584

(22) PCT Filed: Aug. 13, 2004
(86) PCT No.: PCT/EP04/51793

§ 371 Date: Feb. 12, 2007

(51) Int. Cl. A61K 31/716; A61K 31/353; A61K 31/35; A61K 31/375; A61K 8/73

(52) U.S. Cl. 424/70.13; 514/54; 514/548; 514/460; 514/474; 514/454; 514/729

(57) ABSTRACT

Disclosed is the use of β-(1,3)-β-(1,4)-glucan (a) as carrier for carrying a chemical substance through the stratum corneum into deeper layers of the skin and/or (b) for improving the penetration abilities of the chemical substance through the stratum corneum into deeper layers of the skin.

This invention primarily relates to the use of β-(1,3)-β-(1,4)-glucan (a) as carrier for carrying a chemical substance through the stratum corneum into deeper layers of the skin and/or (b) for improving the penetration abilities of the chemical substance through the stratum corneum into deeper layers of the skin.

Oat derived β-(1,3)-β(1,4)-glucan is preferably used in the context of the present invention and is hereinafter also referred to as “Oat Glucan”.

Corneocytes and intercellular lipids constitute the structure of the uppermost layer of the skin, the stratum corneum. It shares few properties with other biological barriers such as cellular membranes. The corneocytes are enclosed by a highly chemically resistant crosslinked envelope. The intercellular lipids are particularly suited to render the barrier impermeable. There are however, three putative pathways, i.e. appendageal (along hair follicles), transcellular, or intercellular. The latter pathway is considered the most prevailing one. Typically, in order to penetrate the stratum corneum to any appreciable extend, a molecule would have to be less than 1,000 Daltons in size, uncharged, and have to have a logP of between −1 and 3. (Schaefer, H. et al. (1996) Skin Barrier (Karger) pp. 43, pp. 116, pp. 151).

Gums are either hydrophobic or hydrophilic substances of molecular weight ranging from 10,000 to 50,000,000 Daltons. At low concentrations they are capable of forming gels, highly viscous suspensions or solutions when added to an appropriate solvent system. Gums commonly used in cosmetics, medicine or food include agar, algin, aloe, beta glucan, carrageenan, cellulose derivatives, gellan, guar gum, gum arabic, locust bean gum, pectin, pullulan, starches, xanthan (see Whistler, R. L. (1993) Industrial Gums: Polysaccharides and their derivatives Eds. Whistler R L and BeMiller J. N. (Academic Press) pp 2).

Glucans are homopolysaccharides consisting of glucose only. Glucans are distinctive polymers of glucose differentiated from other polymers by not only their source but also their physicochemical properties. However, different stereochemical conformations exist, since it is possible to link the glucose molecules in different ways. Hence, glucans are a diverse group of compounds with differing chemical, physical, and functional properties. The influence of the chemical structure of polysaccharides on their properties can be appreciated by comparing the common properties of some common homoglucans. Thus, Dextran, a (1,6)-α-glucan, with a small degree of branching, is extremely water soluble and nongel forming. Amylose, a (1,4)-β-D-glucan, is sparingly soluble in water and can form rigid thermo-reversible gels. Cellulose, a (1,4)-β-D-glucan, is water insoluble and highly crystalline compared to other polysaccharides. Oat β-(1,3)-α-(1,4)-glucan is classified as a viscous gum, (see Wood, P J 15 (1993) Oat Bran Ed P J Wood (American Association of Cereal Chemists, Inc. St. Paul, Minn.)). Unmodified oat β-(1,3)-β(1,4)-glucan forms highly viscous solutions in water at concentrations >0.75% wt. At concentrations >1.2% wt. the solutions have the consistency of a thick hydrogel.

Cereal β-(1,3)-β-(1,4)-glucans are structural polysaccharides present in the cell wall of cereals like barley and oat, among others. Oat β-(1,3)-β-(1,4)-glucan, is recognised by the U.S. FDA as an agent that may aid the prevention of heart disease. In 1997, the FDA allowed oat products to make a health claim.

Glucans derived from yeast, fungi, certain bacteria and genetically engineered bacteria are of significantly different molecular structure and have different physical and chemical properties compared to oat glucan.

The structure of Oat Glucan indicating the 1,3- and 1-4-linkages is as follows:

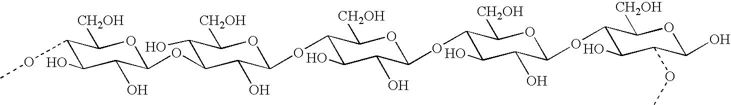

Oat Glucan is a water-soluble polysaccharide consisting of linear chains of glucose units with 1-3 and 1-4 linkages and has a mean molecular weight in the range of 500,000-1,000,000, typically of about 1 million.

Methods for formulating cereal beta glucan compositions that retard the natural gel forming properties of hydrocolloids and remain free flowing liquids are disclosed in WO 99/61480. This documents provides for a simple and efficient method of formulating and producing stable solutions of beta-glucan. A biological, zwitterionic buffer is utilized during the purification process to retard gelation and/or precipitation of the beta-glucan upon cooling. The resulting beta-glucan preparation can be used directly or stored for future use.

Oat Glucan preferably used in the present invention can be obtained according to the process described in WO 99/61480. Such Oat Glucan is a liquid commercially available from Symrise Inc. or Symrise GmbH & Co. KG.

An aqueous 1% wt. solution of such Oat Glucan gives a viscous clear to opal product (at 25° C. such a solution shows a viscosity of 500-1,000 mPas, measured with a rotational, shear-type viscosimeter such as the Brookfield Syncro-Lectric or the Haake Rotovisco).

EP 0 476 063 describes pharmaceutical compositions comprising a drug chemically bound to or being contained within whole beta-glucan particles. In particular a whole beta-glucan drug delivery vehicle that non-specifically enhances the immune response, and is safe for human use, is taught. A drug is incorporated into a whole beta-glucan microparticle, and the combination is administered to an individual. The beta-glucan vehicle allows sustained release of the drug component while simultaneously enhancing the effectiveness of the drug by boosting the individual's endogenous immune response.

WO 96/14873 discloses a glucan composition containing a beta-1,3-glucan covalently attached to a bioactive agent. The beta-1,3-glucan is attached to the bioactive agent by means of a hydrolyzable covalent linkage to form a glucan/agent product. Also disclosed are methods relating to said product, including a method for the treatment of a pathogen capable of invading or colonizing phagocytic cells, and a method for delivering an antigen to a phagocytic cell.

U.S. Pat. No. 5,676,967 discloses a wound dressing for covering a wound of the body, providing slow release of a combination of collagenic protein and oligosaccharide, enhancing vapor transmission from the wound, and enhancing healing. It comprises an aqueous combination of collagen and oligosaccharide coated on a mesh surface and dehydrated to a low moisture content. The mesh netting used has holes or openings, and the structure of the netting permits a solution of oligosaccharide such as glucan and a collagen to impregnate and fill the openings. The impregnated netting is then dehydrated and oligosaccharide and collagen are deposited and adhere to the fibers. An aqueous based mixture of the oligosaccharide and collagen for impregnating the mesh netting may contain about 1-10 percent oligosaccharide and 1-15 percent collagen. The mixture is applied to the mesh netting to substantially impregnate it. An aqueous-based solution of oat-derived beta-D-glucan and bovine collagen containing Type I and Type III collagens was prepared and the mesh netting impregnated therewith.

EP 1 046 394 is directed to compositions and their use for delivering compounds into a cell. In a preferred embodiment, the compositions comprise, in combination with the compound to be delivered, an organic halide, a targeting ligand, and a nuclear localization sequence, optionally in the presence of a carrier. In case the composition comprises a carrier or stabilizing materials, a large number of polymers may be used, inter alia glucans are suitable. The compositions are particularly suitable for the treatment of inflammatory diseases.

WO 01/87255 relates to an external application having enhanced skin absorbency of the active agents by using protease stabilized by beta-1,3-glucan branched with beta-1,6-linkage as an agent for enhancing the skin absorption. Stabilization of the protease is achieved by a chemical reaction in order to covalently bind the protease to the beta-glucan, the resulting product is then able to enhance the skin absorption of the active agents.

WO 03/054077 teaches the use of beta glucan as a film forming delivery system for controlled delivery of actives into an aqueous system, i.e. the mouth cavity. Cereal β-(1,3)-β-(1,4)-glucan is used as a film or coating agent to produce clear, edible, biodegradable, delivery, lubricating, and protecting agents. The β-(1,3)-β-(1,4)-glucan forms a matrix to sequester other materials, such as pharmaceutical, medical and therapeutic agents, flavours, fragrances. The technology has applications to essential oils and non-aqueous materials that are rendered deliverable by the β-(1,3)-β-(1,4)-glucan. The β-(1,3)-β-(1,4)-glucan films described may be consumed whereby they dissolve in the mouth in a controlled manner and may be used for the delivery of pharmaceutical, medical or confectionery products.

There is no mention in prior art suggesting the use of β-(1,3)-β-(1,4)-glucan (and preferably Oat Glucan) as (a) as carrier for carrying a chemical substance through the stratum corneum into deeper layers of the skin and/or (b) for improving the penetration abilities of said chemical substance through the stratum corneum into deeper layers of the skin.

The present invention is based on the surprising finding that despite the general scientific teaching according to which in order to penetrate the stratum corneum to any appreciable extend, a molecule would have to be less than 1,000 Daltons in size, uncharged, and have to have a logP of between −1 and 3 (see above), β-(1,3)-β-(1,4)-glucan (and preferably Oat Glucan) is capable of penetrating the stratum corneum. It was likewise surprising that β-(1,3)-β-(1,4)-glucan (a) can act as a carrier for chemical substances which on its own cannot penetrate the stratum corneum and (b) can improve the penetration abilities of chemical substances through the stratum corneum into deeper layers of the skin. In particular it has been found that Oat Glucan can—despite its high molecular weight—penetrate through the stratum corneum into intact human skin. In addition Oat Glucan is able to form a complex with actives such as cosmetical, therapeutical and/or pharmaceutical actives without being covalently bound to these actives.

These findings are in particular of interest for the delivery of actives through the stratum corneum into the lower layers of the epidermis and beyond of intact, unharmed human skin (delivery for cosmetic purposes).

In the use according to the present invention, and likewise in methods and compositions according to the present invention, which will be discussed in more detail below, the following embodiments are preferred:

In preferred embodiments the β-(1,3)-β-(1,4)-glucan is not covalently bound to the chemical substance. This allows for an effective release of the chemical substance in the skin. Preferably, the chemical substance and β-(1,3)-β-(1,4)-glucan form a complex.

As mentioned before, the β-(1,3)-β-(1,4)-glucan is preferably prepared from oat. Oat Glucan can be prepared according to the methods described above.

The β-(1,3)-β-(1,4)-glucan preferably has a mean molecular weight of 10,000 to 5,000,000 g/mol, more preferably a mean molecular weight of 100,000 to 1,500,000 g/mol. In particular Oat Glucan having such mean molecular weight has surprisingly been found to be able to penetrate the stratum corneum.

The chemical substance is a preferably a cosmetical active agent.

The chemical substance (cosmetical active) is preferably a compound with a mean molecular weight ranging from 200 to 1,000,000. Such compound can be combined with β-(1,3)-β-(1,4)-glucan (preferably in an preferred embodiment as described above) and be carried through the stratum corneum to deeper layers of the skin.

The chemical substance favorably has a logP between −4 to 5.

The chemical substance (cosmetical active) is preferably a polymer, protein, or peptide or a mixture thereof, preferably a protein or peptide of either natural or synthetic origin. Such compounds regularly have very limited skin penetration abilities.

In preferred embodiments the chemical substance (cosmetical active) is selected from L-ascorbic acid, L-ascorbic acid derivatives, kojic acid, kojic acid derivatives, xanthin derivatives, bisabolol, vitamine E and vitamine E derivatives (e.g. tocopherolacetate).

The use is preferably non-therapeutic.

Further preferred embodiments of the use according to the present invention are described in the following parts of the description, including the examples, and in the attached claims.

The present inventions also relates to a method of delivering a chemical substance through the stratum corneum to deeper layers of the skin, the method comprising the following steps:
mixing the chemical substance with an effective amount of β-(1,3)-β-(1,4)-glucan,
topically applying the resulting mixture to the skin.

Regarding further preferred embodiments we particularly refer to the preferred embodiments for the use according to the present invention.

The present invention further relates to a topical composition comprising a mixture of (a) β-(1,3)-β-(1,4)-glucan and (b) at least one cosmetical, therapeutical and/or pharmaceutical active, said β-(1,3)-β-(1,4)-glucan and said active not being covalently bonded to each other, wherein the amount of β-(1,3)-β-(1,4)-glucan is chosen such that it (a) can act as carrier for carrying the chemical substance through the stratum corneum into deeper layers of the skin and/or (b) improves the penetration abilities of the chemical substance through the stratum corneum into deeper layers of the skin.

Again, regarding preferred embodiments we particularly refer to the preferred embodiments for the use according to the present invention.

Favourably, in the composition of the present invention the active is not a collagenic protein.

According to a related further aspect of the present invention β-(1,3)-β-(1,4)-glucan (favourably Oat Glucan) is used for producing a topical composition according to the present invention.

The present invention relates to the use of Oat Glucan as delivery agent for the delivery of one or more chemical substances like cosmetical, therapeutical and/or pharmaceutical actives through the stratum corneum into the epidermis and even into deeper layers of the skin.

In combinations of (and favourably complexes consisting of) Oat Glucan and actives, such as cosmetical, therapeutical and/or pharmaceutical actives, Oat Glucan can be considered a delivery agent for said actives and may be used for the delivery of said actives through the stratum corneum into deeper layers of the skin. This is particularly an advantage when said complex is used in topical applications. For best results, the combination/complex is obtained by premixing or homogenizing Oat Glucan and active before subsequent processing and incorporation into a topical (cosmetic) composition.

Preferred is a mixture (preferably a complex) of Oat Glucan and active wherein the ratio of Oat Glucan to active is ranging from 1,000:1 to 1:1,000, preferably ranging from 100:1 to 1:100, even more preferred ranging from 30:1 to 1:30.

A mixture of β-(1,3)-β-(1,4)-glucan (preferably Oat Glucan) and active (or other chemical substance) as used according to the present invention preferably is incorporated into a topical (cosmetic) composition in an amount ranging from 0.005 to 10% by weight, more preferably 0.01 to 5% by weight, based on the total weight of the topical (cosmetic) composition.

Topical (cosmetic) compositions according to the present invention can comprise (cosmetic and/or dermatological) auxiliaries and additives, as are usually used in such preparations, e.g., sunscreens/uv-absorbers (e.g., organic or, inorganic light filter substances, micropigments), preservatives, bactericides, fungicides, virucides, ingredients which have a cooling action, plant extracts, antiinflammatory active ingredients, substances which accelerate wound healing (e.g. chitin or chitosan and derivatives thereof), film-forming substances (e.g. polyvinylpyrrolidones or chitosan or derivatives thereof), customary antioxidants, vitamins (e.g. vitamin C and derivatives, tocopherols and derivatives, vitamin A and derivatives), 2-hydroxycarboxylic acids (e.g. citric acid, malic acid, L-, D- or dl-lactic acid), skin lighteners (e.g. kojic acid, hydroquinone or arbutine, ascorbic acid, ascorbic acid derivatives, hydroquinone, hydroquinone derivatives, sulfur-containing molecules, such as, for example, glutathione or cysteine or other synthetic or natural active ingredients for skin lightening, it being possible for the latter to be used also in the form of an extract from plants, such as, for example, bearberry extract and rice extract), skin colorants (e.g. walnut extracts or dihydroxyacetone), perfumes, fragrance compounds, antifoams, dyes, pigments which have a coloring action, thickeners, surface-active substances, emulsifiers, emollients, humectants and/or moisturizers (e.g. glycerol or urea), fats, oils, unsaturated fatty acids or derivatives thereof (e.g. linoleic acid, (α-linolenic acid, γ-linolenic acid or arachidic acid and the natural or synthetic esters thereof in each case), waxes or other customary constituents of a cosmetic or dermatological formulation, such as alcohols, polyols, polymers, foam stabilizers, electrolytes, organic solvents, silicone derivatives or chelating agents (e.g., ethylenediaminetetraacetic acid and derivatives).

The amounts of (cosmetic and/or or dermatological) auxiliaries or additives to be used in each case can easily be determined by simple exploratory experiments by the person skilled in the art, depending on the nature of the product in question.

The topical (cosmetic) composition according to the present invention can additionally comprise UVA and/or UVB filter substances. The total amount of UV filter substances preferably may be from 0.1 to 30% by weight, more preferably from 0.5 to 10% by weight, based on the total weight of the composition, giving, for example, sunscreens for skin and hair. Examples of UV filter substances which can be used are 3-benzylidenecamphor derivatives (e.g., 3-(4-methylbenzylidene)-dl-camphor), amino-benzoic acid derivatives (e.g., 2-ethylhexyl 4-(N,N-dimethylamino)benzoate or methyl anthranilate), 4-methoxycinnamates (e.g., 2-ethylhexyl p-methoxycinnamate or isoamyl p-methoxycinnamate), benzophenones (e.g., 2-hydroxy-4-methoxybenzophenone), mono- or polysulphonated UV filters [e.g., 2-phenylbenzimidazole-5-sulphonic acid, sulisobenzones or 1,4-bis(benzimidazolyl)-benzene-4,4′,6,6′-tetrasulphonic acid and 3,3′-(1,4-phenylenedimethylidene)-bis-(7,7-dimethyl-2-oxo-bicyclo-[2,2,1]heptane-1-methanesulphonic acid) and salts thereof), salicylates (e.g., 2-ethylhexyl salicylate or homomethyl salicylate), triazines (e.g., 2,4-bis-[4-(2-ethylhexyloxy)-2-hydroxyphenyl]-6-(4-methoxyphenyl)-1,3,5-triazine, bis-(2-ethylhexyl) 4,4′-([6-([(1,1-dimethylethyl)-aminocarbonyl]phenylamino)-1,3,5-triazin-2,4-diyl]dimino)bisbenzoate), 2-cyanopropenoic acid derivatives (e.g., 2-ethylhexyl 2-cyano-3,3-diphenyl-2-propenoate), dibenzoyl derivatives (e.g., 4-tert.-butyl-4′-methoxydibenzoylmethane), polymer-bonded UV filters (e.g., polymer of N-[2-(or 4)-(2-oxo-3-bomylidene)methyl]-benzylacrylamide) or pigments (e.g., titanium dioxides, zirconium dioxides, iron oxides, silicon dioxides, manganese oxides, aluminium oxides, cerium oxides or zinc oxides).

The lipid phase in the topical (cosmetic) compositions according to the present invention can advantageously be chosen from the following groups of substances: mineral oils (advantageously paraffin oil), mineral waxes, hydrocarbons (advantageously squalane or squalene), synthetic or semisynthetic triglyceride oils (e.g., triglycerides of capric or caprylic acid), natural oils (e.g., castor oil, olive oil, sunflower oil, soya oil, peanut oil, rapeseed oil, almond oil, palm oil, coconut oil, palm kernel oil, borage oil seed oil and the like), natural ester oils (e.g., jojoba oil), synthetic ester oils (preferably esters of saturated and/or unsaturated, linear and/or branched alkanecarboxylic acids carrying from 3 to 30 carbon atoms with saturated and/or unsaturated, linear and/or branched alcohols having from 3 to 30 carbon atoms and esters of aromatic carboxylic acids with saturated and/or unsaturated, linear and/or branched alcohols having from 3 to 30 carbon atoms, in particular, chosen from the group consisting of isopropyl myristate, isopropyl stearate, isopropyl palmitate, isopropyl oleate, n-butyl stearate, n-hexyl laurate, n-decyl laurate, isooctyl stearate, isononyl stearate, isononyl isononanoate, 2-ethylhexyl palmitate, 2-ethylhexyl laurate, 2-hexyldecyl stearate, 2-octyldecyl palmitate, oleyl oleate, oleyl erucate, erucyl oleate, erucyl erucate, and synthetic or natural mixtures of such esters), fats, waxes and other natural and synthetic fatty substances, preferably esters of fatty alcohols with alcohols of low carbon number (e.g., with isopropanol, propylene glycol or glycerol) or esters of fatty alcohols with alkanoic acids of low carbon number or with fatty acids, alkyl benzoates (e.g., mixtures of n-dodecyl, n-tridecyl, n-tetradecyl and n-pentadecyl benzoate), and cyclic or linear silicone oils (such as, for example, dimethylpolysiloxanes, diethylpolysiloxanes, diphenylpolysiloxanes, and mixed forms thereof).

The aqueous phase of the topical (cosmetic) compositions according to the present invention optionally, advantageously comprises alcohols, diols or polyols of low carbon number, and ethers thereof, preferably ethanol, isopropanol, propylene glycol, glycerol, ethylene glycol, ethylene glycol monoethyl or monobutyl ethers, propylene glycol monomethyl, monoethyl or monobutyl ethers, diethylene glycol monomethyl or monoethyl ethers and analogous products, and also alcohols of low carbon number, e.g., ethanol, isopropanol, 1,2-propanediol, glycerol, and also α- or β-hydroxy acids, preferably lactic acid, citric acid or salicylic acid, and also emulsifiers, which may be advantageously chosen from the group consisting of ionic, nonionic, polymeric, phosphate-containing and zwitterionic emulsifiers, and, in particular one or more thickeners, which may advantageously be chosen from the group consisting of silicon dioxide, aluminium silicates, such as, for example, bentonites, polysaccharides and derivatives thereof, e.g., hyaluronic acid, guar flour, xanthan gum, hydroxypropylmethylcellulose or allulose derivatives, particularly advantageously from the group of polyacrylates, preferably, a polyacrylate from the group of so-called Carbopols, in each case individually or in combination, or from the group of polyurethanes.

For use, the topical (cosmetic) compositions according to the present are applied to the skin and/or hair.

### EXAMPLES

Unless indicated otherwise all figures and percentages given are by weight.

Oat Glucan as used in the Examples can be obtained according to the process described in WO 99/61480. Oat Glucan as used in the Examples is commercially available from Symrise Inc. or Symrise GmbH & Co. KG.

### Example 1

Phacocell® was used for the Oat Glucan penetration study. In a pilot-study the method was adapted, methodical errors, like presence of fungi etc. were eliminated. The method was developed as to match actual in vivo conditions as best as possible, in view of the variability of the fluorescence staining technique.

1. Method

1.1 Skin Specimens

Abdominal human skin was surgically removed and cut to size to fit the chamber. The skin sections were stored under liquid nitrogen and sterilized by gamma-radiation with 25 KGy overnight. This treatment detroys all fungi, which could interfere with the analysis. After radiation, the skin samples were defrosted and inspected macroscopically for integrity before use in order to avoid leaks. The skin specimen were inspected for leaks again after staining with fluorescent stain.

1.2 Target Variables, Preparations, Dosage

The main goal of the study was to determine the glucan concentration on top of the skin and in the layers of the skin (stratum corneum, epidermis, dermis, and subcutis). The applied dosage of 5 mg/cm² corresponds realistic in vivo amounts. The fluorescence technique allows semi-quantitative results, which were determined from slides. The preparations were applied uniformly to the skin surface using a micro dose applicator (volume, graduation weight based).

1.3. Phacocell®

The Phacocell® cell has a chamber with the volume of 20 ml, filled with an acceptor medium, in which the active is well soluble. The medium was set at 36° C. and circulated continuously. The chamber was kept free of air bubbles during filling in order to guarantee complete and uncompromised contact with the tissue. Pressure compensation, in and outside of the chamber and constant humidity was provide by ventilation. The skin specimen was conditioned with respect to surface temperature (32° C.) and moisture content (65 RU corneometer units) via a ventilation channel. The desired conditioning was achieved by preheating the medium, by controlling the heating plate in the base of the chamber and by controlling the air tubes, and by adjusting the flow speed of the air. These parameters were constant during each of the series of experiments. The skin specimens from surgery were free of fat tissue. A macroscopic and physical inspection of the suitability of the skin specimen was carried out (before application pressure tested for leaks, and after 8 h by colordetection for leaks). The area of application was 10 cm² for all samples. The skin specimen was supplied with nutrients via the uniformly circulating nutrient medium, which was in contact with the bottom surface of the skin sample. The skin temperature was monitored with temperature sensors, and the moisture content with a corneometer. The settings of the air flow across the skin's upper surface precluded hydration of the skin specimens and thus rendered the experimental conditions non-occlusive.

1.4. Fluorescence Microscopy

Sophisticated microscopes and numerous accessories have been developed based on the principals of fluorescence. Epi-fluorescence, or incident light fluorescence, has now become the method of choice in many applications. The phenomenon of fluorescence is based on a light emission that continues only during the absorption of the excitation light by a chromophore or other conjugated molecule, which is capable of emitting secondary fluorescence.

Excitation and Emission Fundamentals—fluorochromes have unique and characteristic spectra for absorption (usually similar to excitation) and emission. The visible emission spectra and the associated phenomena are utilized in fluorescence microscopy.

Light Sources—In order to generate enough excitation light intensity to furnish secondary fluorescence emission capable of detection, powerful light sources were used. These xenon arc (burner) lamps, which produce high-intensity illumination was powerful enough to image faintly visible fluorescence specimens.

Filter Cubes—Microscope manufacturers (LEIKA) provide proprietary filter cubes that contain a combination of dichroic mirrors and filters capable of exciting fluorescent chromophores and diverting the resulting secondary fluorescence to the eyepieces or camera tube. A LEIKA fluorescence microscope was used with an exciter filter ranging between 400-500 nm with a peak at 440 nm and a barrier filter of 500-520 nm for optimum results.

1.5. Skin Swab Samples

The skin swab samples were taken with cotton gauze swabs from the skin surface after 8 hours had elapsed: 1^{st} and 3^{rd} swab moistened with 0.2 ml 70% methanol/H₂O, 2^{nd} and 4^{th} swab dry.

1.6. Main Objective: Determination of Oat Glucan Penetration into the Skin

The presence of Oat Glucan in the stratum corneum and in the other skin tissues was detected by microscope. The cleaned skin samples of the Phacocell® chamber were separated with punch-biopsies and immediately deep frozen, cut into thin slices of 15 μm in width. The skin was cut against the penetratino grade, i.e. from the dermis towards the stratum corneum. The slices were air dried and not fixed by any fluid. Fixation with fluids can cause a dilution and/or displacement of Oat Glucan. The proceeding of fluorescence staining was developed by technical service of REMEL Industry, BACTIDROP™. Calcofluor White is recommended for use in qualitative procedures as a rapid, non-specific fluorochrome stain for the initial microscopic detection of fungal elements and can be used for the detection of Oat Glucan molecules as well. Calcofluor white is a non-specific fluorochrome with the ability to bind to cellulose and chitin. Upon excitation with longwave ultraviolet light, this compound functions to delineate the cell walls of cellulose-containing organisms.

1.7. Procedure: Use of BACTIDROP™

A fluorescent microscope with an exciter filter ranging between 400-500 nm with a peak of 440 nm and a barrier filter of 500-520 nm is recommended for optimum results with calcofluor white.

1.8. Controls

Untreated skin was subjected to the same regime in the Phacocell®—No fluorescence staining was detectable.
   1.9. Data Processing—Quality Control The data were entered centrally into the computer (SAS Modul®/Oracle®), following checks for plausibility and quality. Statistical evaluation was carried out by the statistics software package SAS/Statistica®.
   1.10 Results
   Fluorescence—Densitometry

The detection of Oat Glucan in skin tissue after 8 hours indicates an uptake into the horny layer and the epidermis. The degree of fluorescent staining suggested that there were high concentrations of glucan in the horny layer and the epidermis. Levels detected in the dermis and subcutis were lower (Table 1). The different glucan concentrations among the tested samples (sample 1=5% Oat glucan, sample 2=50% Oat glucan) did not translate into proportional concentration in the tissue. The measurement and procedure of staining had an error of 2-3% in tissue. Overall the results show the significant penetration of Oat Glucan into the epidermis.

TABLE 1
Detection of Oat Glucan in the different
layers of the skin after 8 h

| | Sample 1 | | Sample 2 | | Control | |
|---|---|---|---|---|---|---|
| Mean Deposition | [%] | Stdv. | [%] | Stdv. | [%] | Stdv. |
| Stratum corneum | 8.7 | 1.2 | 12.8 | 1.9 | 0.6 | 0.2 |
| Lower layers of | 5.9 | 1.3 | 11.6 | 2.0 | 0.8 | 0.2 |
| Epidermis | | | | | | |
| Dermis | 2.4 | 0.5 | 4.1 | 1.1 | 0.6 | 0.1 |
| Subcutis | 1.4 | 0.5 | 1.5 | 0.4 | 0.9 | 0.1 |
| % recovery | 18.4 | 1.7 | 29.9 | 3.0 | | |

Stdv.: standard deviation

### Example 2

Lotion (W/O)

| Phase | Ingredient | INCI | % wt. |
|---|---|---|---|
| A | Lanette O | Cetearyl Alcohol | 1.80 |
| | Dracorin 100 s.e.P. | Glyceryl Stearate, PEG-100 | 6.00 |
| | | Stearate | |
| | Paraffin Oil (White Oil) | Mineral Oil | 5.00 |
| | Cetiol 868 | Octyl Stearate | 2.00 |
| | Cetiol J600 | Oleyl Erucate | 2.00 |
| | Stearic Acid (Stearin | Stearic Acid | 1.00 |
| | L2 S.M. Luxus) | | |
| B | Propylen Glycol | Propylene Glycol | 3.50 |
| | Water | Aqua | 73.00 |
| | Dragocid liquid | Phenoxyethanol, Methylparaben, | 0.70 |
| | | Ethylparaben, Butylparaben, | |
| | | Propylparaben, Iso-butylparaben | |
| C | β-Glucan/Whey Extract | β-Glucan + Whey Extract (25:1) | 4.8 |
| | (25:1) | | |
| D | Perfume oil | Parfum (Fragrance) | 0.2 |

Preparation Method

Heat phase A and phase B separately to 80° C. Add B to A and homogenize for about 5 min. Homogenize phase C by mixing, by weight, 25 parts Oat Glucan and one part Whey Extract at ambient temperature for about 2 minutes. Cool down the mixture of parts A and B to about 40° C., then add homogenized phase C and subsequently phase D. After homogenization of the topical composition cool down to ambient temperature.

1. A method of carrying a chemical substance through the stratum corneum into deeper layers of the skin and/or improving the penetration abilities of a chemical substance through the stratum corneum into deeper layers of the skin comprising the step of providing a mixture of β-(1,3)-β-(1,4)-glucan and said chemical substance, wherein β-(1,3)-β-(1,4)-glucan is provided in an amount sufficient for acting as carrier for carrying said chemical substance through the stratum corneum into deeper layers of the skin and/or for improving the penetration abilities of said chemical substance through the stratum corneum in to deeper layers of the skin.

2. A method according to claim 1, wherein the β-(1,3)-β-(1,4)-glucan is not covalently bound to the chemical substance.

3. A method according to claim 1, wherein the β-(1,3)-β-(1,4)-glucan is prepared from oat.

4. A method according to claim 1, wherein the β-(1,3)-β-(1,4)-glucan has a mean molecular weight of 10,000 to 5,000,000.

5. A method according to claim 4, wherein the β-(1,3)-β-(1,4)-glucan has a mean molecular weight of 100,000 to 1,500,000.

6. A method according to claim 1, wherein the chemical substance is a cosmetical cosmetically effective active agent.

7. A method according to claim 1, wherein the chemical substance is a compound with a mean molecular weight ranging from 200 to 1,000,000.

8. A method according to claim 7, wherein the chemical substance has a logP between −4 to 5.

9. A method according to claim 7, wherein the substance is a polymer, protein, or peptide or a mixture thereof.

10. A method according to claim 1, wherein the substance is selected from L-ascorbic acid, L-ascorbic acid derivatives, kojic acid, kojic acid derivatives, xanthin derivatives, bisabolol, vitamin E and vitamin E derivatives.

11. A method according to claim 4, comprising a mixture of (a) β-(1,3)-β-(1,4)-glucan and (b) at least one active ingredient that is cosmetically and/or pharmaceutically effective.

12. A method according to claim 1, comprising a mixture of (a) β-(1,3)-β-(1,4)-glucan and (b) at least one active ingredient that is cosmetically effective.

13. A method of delivering a chemical substance through the stratum corneum to deeper layers of the skin, comprising the following steps:
mixing the chemical substance with an effective amount of β-(1,3)-β-(1,4)-glucan,
topically applying the resulting mixture to the skin.

14. A topical composition comprising a mixture of (a) β-(1,3)-β-(1,4)-glucan and (b) at least one active ingredient that is cosmetically, therapeutically and/or pharmaceutically effective provided that, said β-(1,3)-β-(1,4)-glucan and said active are not covalently bonded to each other, and wherein the amount of β-(1,3)-β-(1,4)-glucan is sufficient to:
(1) act as carrier for carrying the chemical substance through the stratum corneum into deeper layers of the skin and/or
(2) improve the penetration abilities of the chemical substance through the stratum corneum into deeper layers of the skin.

15. A topical composition according to claim 13, wherein the active is not a collagenic protein.

16. A method of formulating β-(1,3)-β-(1,4)-glucan in a form suitable for use as a topical composition according to claim 13 comprising
premixing or homogenizing said mixture
processing and incorporating said mixture into a topical composition.
